(19) United States
(12) Patent Application Publication
(10) Pub. No.: US 2002/0045942 A1
(43) Pub. Date: Apr. 18, 2002

(54) Procedure for repairing damaged discs

(76) Inventor: Michael J. Ham, Fairfiled, CT (US)

Correspondence Address: PERMAN & GREEN, 425 POST ROAD, FAIRFIELD, CT, 06430, US

(21) Appl. No.: 09/908,056

(22) Filed: Jul. 18, 2001

Related U.S. Application Data
(63) Continuation-in-part of application No. 09/690,067, filed on Oct. 16, 2000.

(51) Int. Cl. A61F 2/44

(52) U.S. Cl. 623/17.12; 623/17.16

(57) ABSTRACT

A technique for repairing a damaged intervertebral disc having an outer annulus fibrosus and an inner nucleus pulposus includes steps of introducing through the annulus fibrosus and into the nucleus pulposus a biologically inert thermoplastic elastomer in the liquid state with sufficient pressure to reinflate the damaged disc to its normal undamaged dimensions. The thermoplastic elastomer may be cured at room temperature to a hardness sufficient to support normal postural compressive loads and prevent the disc from returning to its damaged dimensions. The duration of the curing step is tailored to obtain optimal physical properties desired for the cured thermoplastic elastomer. A syringe including a barrel filled with the thermoplastic elastomer, an operating plunger, and a projecting needle are positioned adjacent the damaged disc, the needle inserted through the annulus fibrosus and into the nucleus pulposus, and the plunger operated to inject the thermoplastic elastomer into the nucleus pulposus.

### CROSS-REFERENCE TO RELATED APPLICATIONS

This application is a continuation-in-part of co-pending U.S. Patent Application Ser. No. 09/690,067, filed on Oct. 16, 2000.

### BACKGROUND OF THE INVENTION

1. Field of the Invention

The present invention relates to the repair of damaged discs in an animal or human body and, more particularly, to the introduction of a liquid thermoplastic elastomer to a site to be repaired and tailoring the curing of the thermoplastic elastomer to achieve a desired result.

2. Prior Art

Intervertebral discs are interposed between the adjacent surfaces of the bodies of the vertebrae from the axis to the sacrum, forming the chief bonds of connection between the adjoining vertebral bodies. They vary in shape and thickness in different parts of the vertebral column. Their shape corresponds to the surfaces of the bodies between which they are located except in the cervical region where they are slightly smaller from side to side than the corresponding bodies. Their thickness varies not only in the different regions of the column, but in different parts of the same disc. The intervertebral discs are adherent to thin layers of hyaline cartilage which cover the superior and inferior surfaces of the bodies of the vertebrae.

Each disc is composed of outer laminae of fibrous tissue and fibrocartilage called the annulus fibrosus, and an inner core of soft, gelatinous and highly elastic substance called the nucleus pulposus. The laminae forming the annulus fibrosus are arranged in concentric rings and the outermost consist of ordinary fibrous tissue; those closer to the center are formed of white fibrocartilage. The laminae are not quite vertical in their direction, those near the circumference being curved outward and closely approximated, while those nearest the center curve in the opposite direction, and are somewhat more widely separated. The fibers composing the laminae pass obliquely between the two adjacent vertebrae and are firmly attached to them. Greater stability is achieved in the disc because the fibers of each adjacent lamina pass in opposite directions, interlaced like the limbs of the letter X. This laminar arrangement characterizes the outer half of each fibrocartilage.

A common curse of humankind is a ruptured or herniated disc. The function of the human disc is to maintain separation between the adjacent vertebrae comprising the spinal column and to act like a shock absorber and allow the spinal column to move. A human spinal column has five vertebrae in the lumbar region and seven vertebrae in the cervical region and 12 vertebrae in the thoracic region. The lumbar region is commonly referred to as the lower back and the cervical region is commonly referred to as the neck. The thoracic region is in the middle of the spinal column. The spinal column is the primary structural element of the human skeleton. It is required to carry the compressive load of the upper portion of the body and transmit that load to the lower portion of the body. Consequently, it must have the compressive structural strength needed to perform that role over millions of cycles. Also, the spinal column must support the body under the normal human activities such as bending, turning, stooping over and engaging in various forms of exercise. To accommodate this requirement, the spinal column must be capable of rotational twisting without breaking. The dual role is accommodated by the inter-positioning of a human disc between the adjacent vertebrae in the lumbar region and the cervical region and the thoracic region. The function of the human disc is to provide the compressive strength necessary to avoid having the adjacent vertebrae come in contact with each other. For example, the conventional surgical approach for a ruptured cervical disc is to remove the damaged cervical disc and fuse the space now developed between the adjacent vertebrae with a bone graft. Repair plates for anterior cervical fusion are known in the art. Anterior cervical fusion, however, has the disadvantage of reducing the range of rotational motion, due to the joining of the adjacent vertebrae causing “blocked vertebrae”. Over time the range of motion reduction can be significant if more than one fusion is performed, particularly in the cervical region. Furthermore, it causes degeneration of the disc spaces above and below the levels of fusion, thus often necessitating further surgery on adjacent discs.

A broad range of attempts have been made heretofore to repair herniated discs and many of these have been archived in the patent literature. A number of particularly pertinent examples will now be discussed. For example, U.S. Pat. No. 5,964,807 to Gan et al. discloses methods for repairing damaged or degenerated intervertebral discs which include evacuating tissue from the nucleus pulposus portion of a degenerated intervertebral disc space, preparing hybrid material by combining isolated intervertebral disc cells with a biodegradable substrate, and implanting the hybrid material in the evacuated nucleus pulposus space.

U.S. Pat. No. 5,976,186 to Bao et al. relates to a hydrogel prosthetic nucleus which may be implanted in the nuclear cavity of an intervertebral disc as one or more xerogel rods or tubes which may be partially hydrated. The patent states that the prosthetic nucleus of the invention may be brought to its equivalent water content more rapidly than previously known hydrogel prostheses due to its greater surface area and its ability to retain its shape without the support of a container such as the envelope required in the case of nuclei formed from hydrogel beads.

U.S. Pat. No. 5,824,093 to Ray et al. discloses an elongated prosthetic spinal disc nucleus for implantation deep inside a human disc space. The prosthesis is composed of a hydrogel core configured to imbibe fluids after implant, expanding as it hydrates, and a constraining jacket surrounding the hydrogel core. The jacket is flexible but inelastic, directing the hydrogel core to deform and reform in the minor axis.

U.S. Pat. No. 5,800,549 to Bao et al. discloses a method and apparatus for injecting an elastic spinal implant into a cavity in a spinal disc so as to treat disc degeneration.

U.S. Pat. No. 5,755,797 to Baumgartner discloses an implant consisting of a plurality of elastic plastic support members which adapt to the shape of the cavity of the core region of an intervertebral disc and are introduced into the cavity one by one until the cavity is filled.

U.S. Pat. No. 5,645,597 to Krapiva discloses a method for replacing a nucleus pulposus of an intervertebral disc achieved by removing the nucleus pulposus from the intervertebral disc to create a space defined by the inner wall of the annulus fibrosus. A flexible prosthetic disc is then inserted within the space formerly occupied by the nucleus pulposus and the prosthetic disc is subsequently filled with a gel.

U.S. Pat. No. 5,545,229 to Parsons et al. discloses a biocompatible intervertebral disc spacer which possesses mechanical properties akin to those of the normal disc so as to preserve the normal functions of the spinal motion segment. The desired properties are achieved by varying the hardness of the elastomeric material in its nucleus and annulus.

U.S. Pat. No. 5,246,458 to Graham discloses an artificial intervertebral disc for replacing a damaged disc between two adjacent vertebrae in a human spinal column. A pair of cylindrically shaped members in a vertical stacked relationship and a flexible spacer therebetween are joined together in a ball and socket relationship which provides full rotational movement. The flexible spacer provides the resilient compressive strength necessary to maintain the vertical separation of the adjacent vertebrae.

U.S. Pat. No. 3,875,595 to Froning discloses a collapsible plastic bladder-like prosthesis of the same external form as the nucleus pulposus of an intervertebral disc which has a stem through which liquid and/or plastic is introduced to inflate the prosthesis to natural form. Pressure may be adjusted over a period of time and, when finally determined, the stem is severed.

It was with knowledge of the foregoing state of the technology that the present invention has been conceived and is now reduced to practice.

### SUMMARY OF THE INVENTION

The present invention relates to techniques and compositions for repairing a damaged intervertebral disc having an outer annulus fibrosus and an inner nucleus pulposus, and includes the steps of introducing through the annulus fibrosus and into the nucleus pulposus a biologically inert thermoplastic elastomer precursor in the liquid state with sufficient pressure to reinflate the damaged disc to its normal undamaged dimensions. Thereafter, the thermoplastic elastomer precursor is cured in situ to a hardness sufficient to support normal postural compressive loads and prevent the disc from returning to its damaged dimensions. The duration and conditions of the curing step are tailored to obtain optimal physical properties desired for the cured thermoplastic elastomer with biological safety and comfort. For this purpose, a syringe including a barrel filled with the liquid thermoplastic elastomer precursor, an operating plunger, and a projecting needle is positioned adjacent the damaged disc, its needle inserted through the annulus fibrosus and into the nucleus pulposus, and the plunger operated to inject the liquid thermoplastic elastomer precursor into the nucleus pulposus. Curing may be performed at room temperature. Alternatively, a curing agent may be employed of a type and in an amount to assure that the liquid thermoplastic elastomer precursor will cure and thicken or increase in viscosity to the proper extent. In another instance, radiated energy may be applied to the disc to obtain accelerated curing of the liquid thermoplastic elastomer precursor. Also, intersegmental traction may be applied to the adjoining vertebrates of the damaged disc during the steps of introducing and curing the elastomeric polymer in order to assure that the normal, undamaged, dimensions of the disc are filled.

The invention covers procedures for repairing damaged discs in the neck or back. It covers both the materials and the means of administering them to provide relief from problems involving damaged discs. More specifically, the invention covers the use of thermoplastic elastomers in the repair of discs. By varying the curing of the liquid thermoplastic elastomers, physical properties such as compression strength can be tailored to the body's need whether that be for supporting or cushioning the compression due to gravity. The use of these materials and the nondestructive means for their administration make use of the body's natural structure to return it to normal functioning, thereby reducing the time, expense, and trauma associated with current surgical procedures.

In each instance, this procedure can be tailored to the patients' needs by varying the degree of curing and/or the thermoplastic elastomer's molecular weight and viscosity and coefficient of friction after curing. The advantage of employing this technology is that it is inherently less invasive and damaging to surrounding tissues than conventional surgery. This promotes more rapid healing and, because it builds on the undamaged portions of the existing discs, results in a more natural, better functioning, system than the methods currently employed.

In a particularly beneficial application of the invention, a low viscosity, liquid thermoplastic elastomer precursor is injected, perhaps with a curing agent, into compressed or ruptured discs in the neck or spine. The injection should ideally be made laterally on the side of the disc that is most compressed so as to avoid the spinal cord and nerve roots and so as to deposit the material into the space where it is most needed.

The liquid thermoplastic elastomer precursor and the curing agent should be biologically inert, for example, a silicone compound to avoid triggering an auto-immune response (or rejection). The curing process should proceed at normal body temperature within a reasonable amount of time for immobilizing the patient and without any significant exothermic reaction. The positioning of the injection would be facilitated using non-invasive systems such as ultrasound, fluoroscopy, or computer-aided imaging. These same systems or radioactive tracers in the material injected into the cavity could be used to ensure that the space is neither over- or under-inflated.

The needle used to inject the material through the cartilaginous fibers of the disc (the annulus fibrosus) would be left in place until curing proceeded to the point that the material would not leak out of the small hole used for the injection. The injection would be made under pressure while the patient was immobilized. Traction would be used during the injection and curing process to ensure that the disc cavity was restored to its normal, non-compressed dimensions.

In the event a patient experiences a ruptured disc, it may be desirable to remove any of the nucleus pulposus which has extended through and beyond the annulus fibrosus before initiating the procedure of the invention.

A primary feature, then, of the present invention is the provision of a technique for the repair of damaged discs in an animal or human body.

Another feature of the present invention is the provision of such a technique according to which a liquid thermoplastic elastomer precursor is introduced to a site to be repaired and the curing and thickening of the thermoplastic elastomer to a non-fluid condition is tailored to achieve a desired result.

Still another feature of the present invention is the provision of such a technique according to which the liquid thermoplastic elastomer is introduced by means of a syringe.

Yet another feature of the present invention is the provision of such a technique according to which the liquid thermoplastic elastomer is cured at room temperature.

Still a further feature of the present invention is the provision of such a technique according to which a curing agent of a type and in an amount is introduced with the thermoplastic elastomer precursor to assure that the thermoplastic elastomer will cure to a hardness sufficient to support normal postural compressive loads and thereby prevent the disc from returning to its damaged dimensions.

Yet a further feature of the present invention is the provision of such a technique which applies radiated energy to the disc to obtain accelerated curing of the thermoplastic elastomer.

Still another feature of the present invention is the provision of such a technique which applies intersegmental traction to the adjoining vertebrae of the damaged disc during the introduction and curing steps for the thermoplastic elastomer.

Yet another feature of the present invention is the provision of such a technique which uses a computerized navigation system for positioning the needle of the syringe and for injecting the uncured liquid thermoplastic elastomer into the nucleus pulposus, then for monitoring the progress of the injection operation to avoid the possibility of over-inflating the nucleus pulposus in one instance and under-inflating the nucleus pulposus in another instance.

Still a further feature of the present invention is the provision of such a technique which uses a non-destructive soft-tissue monitoring system for positioning the needle and for injecting the uncured liquid thermoplastic elastomer into the nucleus pulposus and also uses the non-destructive soft-tissue monitoring system for monitoring the progress of the curing step to avoid the possibility of over-inflating the nucleus pulposus in one instance and under-inflating the nucleus pulposus in another instance.

Other and further features, advantages, and benefits of the invention will become apparent in the following description taken in conjunction with the following drawings. It is to be understood that the foregoing general description and the following detailed description are exemplary and explanatory but are not to be restrictive of the invention. The accompanying drawings which are incorporated in and constitute a part of this invention, illustrate one of the embodiments of the invention, and together with the description, serve to explain the principles of the invention in general terms. Like numerals refer to like parts throughout the disclosure.

### BRIEF DESCRIPTION OF THE DRAWINGS

The foregoing aspects and other features of the present invention are explained in the following description, taken in connection with the accompanying drawings, wherein:

FIG. 1 is a top plan view of a body of a vertebra with a cross section through an invertebral disc to be repaired by the technique of the invention; and

FIG. 2 is a cross section view taken generally along line 2-2 in FIG. 1.

### DETAILED DESCRIPTION OF THE PREFERRED EMBODIMENTS

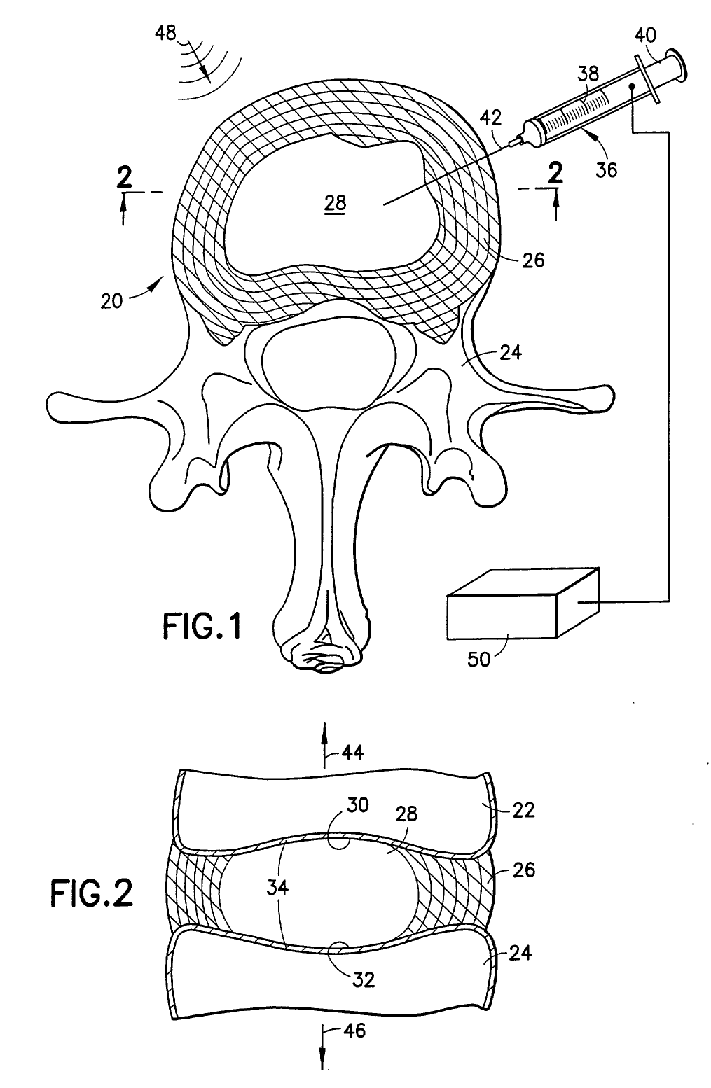

Referring to FIGS. 1 and 2, there are shown cross section views of a formerly damaged, collapsed or herniated, intervertebral disc 20 which is being repaired in accordance with the technique of the present invention. Although the present invention will be described with reference to the embodiments shown in the drawings, it should be understood that the present invention can be embodied in many alternate forms or embodiments. In addition, any suitable additional steps of the method or size, shape or type of elements or materials for performing the method could be used.

As shown in FIGS. 1 and 2, an intervertebral disc 20 positioned between two vertebral bodies 22, 24 includes an intact outer annular region, or annulus fibrosus, 26 of natural tissue and an inner nucleus pulposus 28. The disc 20 functions to permit flexible articulation of the adjacent vertebrae 22, 24 and an internal resistance to flexion (or a bending torque) which lends intrinsic stability to the multisegmented column. The bodies 22, 24 have concave upper and lower surfaces 30, 32 and a layer 34 of cartilage overlies those surfaces.

In many of the known techniques for repairing damaged intervertebral discs, as noted above, the nucleus pulposus 28 was removed and replaced with a prosthesis or other material intended to simulate the original nucleus pulposus. In the instance of the invention, whatever remains of the original nucleus pulposus continues to remain. Then, as diagrammatically indicated in FIG. 1, a syringe 36 of known design including a barrel 38, an operating plunger 40, and a projecting needle 42 is positioned adjacent the damaged disc 20. The barrel 38 is filled with a biologically inert curable thermoplastic elastomer, for example, a silicone, in the liquid state and the needle 42 is inserted through the annulus fibrosus 26 and into the nucleus pulposus 28 of the damaged disc. With the patient immobilized, the plunger 40 is then operated to inject the thermoplastic elastomer from the barrel into the nucleus pulposus of the damaged disc. The liquid thermoplastic elastomer is introduced with sufficient pressure to reinflate the damaged disc to its normal undamaged dimensions. Preferably, the injection is made on the side of the disc which has become most compressed. Thereupon, the thermoplastic elastomer is cured to a viscosity and hardness sufficient to support normal postural compressive loads and thereby prevent the intervertebral disc from returning to its damaged dimensions. The hardened injection would then support the annulus fibrosus, helping to prevent future damage from tilting or twisting. The curing process should proceed at normal body temperatures, or room temperature, without excessive exothermic reaction and within a reasonable time for immobilizing the patient, ideally less than one hour. The duration of the cure can be adjusted via a curative agent, concentration thereof, or by varying the polymer to obtain the optimal physical properties desired for the cured thermoplastic elastomer previously introduced into the nucleus pulposus, thereby tailoring the injected material to the particular needs of the patient.

As earlier indicated, the needle 42 used to inject the thermoplastic elastomer through the annulus fibrosus would preferably be left in place until curing proceeded to the point that the material would thicken and not leak out of the small hole used for the injection. Also, traction, as indicated by opposing arrows 44, 46 in FIG. 2 would be used during the injection and curing process to ensure that the disc cavity is restored to its normal, non-compressed dimensions for the procedure. The use of intersegmental traction during the injection procedure and curing of the material assures that the thermoplastic elastomer will fill the void naturally while the patient is free of pain and then set in the required position with the interspace straight and regular. This should result in the return of the nucleus pulposus to its normal spherical shape and size, thus allowing tilting, rotation, and gliding of the joint.

Also, as an alternative, it may be desirable to introduce with the liquid thermoplastic elastomer a curing agent of a type and in an amount to assure that the thermoplastic elastomer will cure to a viscosity and hardness sufficient to support normal postural compressive loads and thereby prevent the disc from returning to its damaged dimensions.

In another instance, in order to obtain accelerated curing of the thermoplastic elastomer, it may be desirable to apply radiated energy such as ultrasound, as indicated by arrow 48, to the disc 20 to obtain accelerated curing of the thermoplastic elastomer.

The selection of suitable or appropriate curable liquid thermoplastic elastomer precursor systems will be apparent to those skilled in the art in the light of the present disclosure. Such systems must be biologically inert and safe, and curable under medically-safe conditions from a liquid, injectable state to a stable thick, viscous gel or semi-solid state, whereby it fills and inflates the damaged disk and is cured to a hardness sufficient to support the normal postural compressive loads and thereby prevent the intervertabral disk from returning to its damaged dimensions.

Known liquid synthetic elastomer precursor systems suitable for use according to the present invention include:

(a) EPDM (ethylene propylene diene monomer) curable liquid pre-polymers commercially-available from Uniroyal Chemical under the trademark Trilene® 175.105 and 177.1210, both curable using peroxide catalysts;

(b) polyurethane curable liquid pre-polymers commercially available from Uniroyal Chemical under the trademarks Adiprene® and Vibrathane®, also curable by using peroxide catalysts;

(c) silicone rubber curable liquid prepolymers commercially-available from Dow-Corning for biomedical applications under the trademarks Silastic® Q7-4840 and Q7-4850, and also curable with peroxide catalysts; and

(d) synthetic curable liquid rubber prepolymers of the styrene butadiene latex type commercially-available from Dow Chemical under the trademarks SB1502-Schkopan and SES-1502S, and curable by radiation, ultrasound or diathermy.

The invention also encompasses the use of a computerized navigation system 50 to perform non-destructive soft-tissue monitoring for positioning the needle and for injecting the uncured thermoplastic elastomer into the nucleus pulposus.

Such soft-tissue monitoring can be performed by ultrasound imaging, fluoroscopes, radioactive tracers, and by other suitable instrumentation.

The system 50 would also have the capability of monitoring the injection progress to avoid the possibility of over-inflating the nucleus pulposus in one instance and under-inflating the nucleus pulposus in another instance.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

What is claimed is:

1. A method for repairing a damaged intervertebral disc defined by an outer annulus fibrosus and an inner nucleus pulposus comprising the steps of:
(a) introducing through the annulus fibrosus and into the nucleus pulposus of the damaged disc a biologically inert thermoplastic elastomer in the liquid state with sufficient pressure to reinflate the damaged disc to its normal undamaged dimensions; and
(b) curing the thermoplastic elastomer to a hardness sufficient to support normal postural compressive loads and thereby prevent the disc from returning to its damaged dimensions.

2. A method as set forth in claim 1 wherein step (a) includes the steps of:
(c) positioning a syringe including a barrel filled with the thermoplastic elastomer, an operating plunger, and a projecting needle adjacent the damaged disc;
(d) inserting the needle through the annulus fibrosus and into the nucleus pulposus of the damaged disc; and
(e) operating the plunger to inject the thermoplastic elastomer from the barrel into the nucleus pulposus of the damaged disc via the needle.

3. A method as set forth in claim 1 including the step of:
(c) adjusting the duration of step (b) to obtain the optimal physical properties desired for the cured thermoplastic elastomer previously introduced into the nucleus pulposus.

4. A method as set forth in claim 1 wherein step (b) is performed at room temperature.

5. A method as set forth in claim 1 wherein step (a) includes the step of:
(c) introducing with the thermoplastic elastomer a curing agent of a type and in an amount to assure that the thermoplastic elastomer will cure to a hardness sufficient to support normal postural compressive loads and thereby prevent the disc from returning to its damaged dimensions.

6. A method as set forth in claim 1 wherein step (b) includes the step of:
(c) applying radiated energy to the disc after step (a) to obtain accelerated curing of the thermoplastic elastomer.

7. A method as set forth in claim 1 including the step of:
(c) applying intersegmental traction to the adjoining vertebrates of the damaged disc during the performance of steps (a) and (b).

8. A method as set forth in claim 2 including the steps of:
(c) using a computerized navigation system for positioning the needle and for injecting the uncured thermoplastic elastomer into the nucleus pulposus; and
(d) using the computerized navigation system for monitoring the progress of step (b) to avoid the possibility of over-inflating the nucleus pulposus in one instance and under-inflating the nucleus pulposus in another instance.

9. A method as set forth in claim 2 including the steps of:
(e) using a non-destructive soft-tissue monitoring system for positioning the needle and for injecting the uncured thermoplastic elastomer into the nucleus pulposus; and
(f) using the non-destructive soft-tissue monitoring system for monitoring the progress of step (b) to avoid the possibility of over-inflating the nucleus pulposus in one instance and under-inflating the nucleus pulposus in another instance.
